SEQUENCE LISTING (33 sequences)

SEQ ID NO: 1 - protein, 15 aa, Influenza A virus
ARDFYDYDVFYYAMD

SEQ ID NO: 2 - DNA, 30 bp, Artificial Sequence
  Synthetic construct
actatctttggtctattatcttgagtcatc

SEQ ID NO: 3 - DNA, 30 bp, Artificial Sequence
  Synthetic construct
tagttgtgtgtgtgttagaccaaagatagt

SEQ ID NO: 4 - DNA, 30 bp, Artificial Sequence
  Synthetic construct
gatgactcaagataaacacacacacaacta

SEQ ID NO: 5 - protein, 10 aa, Artificial Sequence
  Synthetic construct
AGVKDGKLDF

SEQ ID NO: 6 - DNA, 72 bp, Artificial Sequence
  Synthetic construct
acgatctttgttctactgatgcctgactgatccatgttatattgagtgatgtacaaatcggcgtagtgaagc

SEQ ID NO: 7 - DNA, 51 bp, Artificial Sequence
  Synthetic construct
tagttgtgtgtgtgtcatggatcagtcaggcatcagtagaacaaagatcgt

SEQ ID NO: 8 - DNA, 51 bp, Artificial Sequence
  Synthetic construct
gcttcactacgccgatttgtacatcactcaatataaacacacacacaacta

SEQ ID NO: 9 - DNA, 51 bp, Artificial Sequence
  Synthetic construct
actatctttggtctattatcttgtgtcatctatacatcggcgtggtcaatg

SEQ ID NO: 10 - DNA, 30 bp, Artificial Sequence; identical to SEQ ID NO: 3
  Synthetic construct

SEQ ID NO: 11 - DNA, 51 bp, Artificial Sequence
  Synthetic construct
cattgaccacgccgatgtatagatgacacaagataaacacacacacaacta

SEQ ID NO: 12 - DNA, 31 bp, Artificial Sequence
  Synthetic construct
actatctttggtctatttatcttgagtcatc

SEQ ID NO: 13 - DNA, 31 bp, Artificial Sequence
  Synthetic construct
tagcaacacacctatttagaccaaagatagt

SEQ ID NO: 14 - DNA, 31 bp, Artificial Sequence
  Synthetic construct
gatgactcaagataatataggtgtgttgcta

SEQ ID NO: 15 - DNA, 30 bp, Artificial Sequence
  Synthetic construct
actatctttggtcttttatcgtgagtcatc

SEQ ID NO: 16 - DNA, 30 bp, Artificial Sequence
  Synthetic construct
agttgtgtgtgtgattagaccaaagatagt

SEQ ID NO: 17 - DNA, 30 bp, Artificial Sequence
  Synthetic construct
gatgactcacgatatttcacacacacaact

SEQ ID NO: 18 - DNA, 30 bp, Artificial Sequence; identical to SEQ ID NO: 2
  Synthetic construct

SEQ ID NO: 19 - DNA, 30 bp, Artificial Sequence; identical to SEQ ID NO: 4
  Synthetic construct

SEQ ID NO: 20 - DNA, 15 bp, Artificial Sequence
  Synthetic construct
actatctttggtcta

SEQ ID NO: 21 - DNA, 30 bp, Artificial Sequence; identical to SEQ ID NO: 4
  Synthetic construct

SEQ ID NO: 22 - DNA, 30 bp, Artificial Sequence; identical to SEQ ID NO: 3
  Synthetic construct

SEQ ID NO: 23 - DNA, 67 bp, Artificial Sequence
  Synthetic construct
acgctctttgttctactgatgcttgcctgatccatgatatatattgagtgctatttagtttctatca

SEQ ID NO: 24 - DNA, 67 bp, Artificial Sequence
  Synthetic construct
acacacacacaactaagcactcaatatatatcatggatcaggcaagcatcagtagaacaaagagcgt

SEQ ID NO: 25 - DNA, 30 bp, Artificial Sequence
  Synthetic construct
tgatagaaactaaatataatatgcgagcca

SEQ ID NO: 26 - DNA, 30 bp, Artificial Sequence
  Synthetic construct
tggctcgcatattattagttgtgtgtgtgt

SEQ ID NO: 27 - protein, 15 aa, Artificial Sequence
  Synthetic construct
ARDFYGYDVFFYAMD

SEQ ID NO: 28 - protein, 12 aa, Artificial Sequence
  Synthetic construct
WLVFFVIFYFFR

SEQ ID NO: 29 - protein, 17 aa, Artificial Sequence
  Synthetic construct
LFRLIKSLIKRLVSAFK

SEQ ID NO: 30 - protein, 10 aa, Artificial Sequence
  Synthetic construct
PWLKPGDLDL

SEQ ID NO: 31 - protein, 28 aa, Artificial Sequence
  Synthetic construct
RWMVWRHWFHRLRLPYNPGKNKQNQQWP

SEQ ID NO: 32 - protein, 19 aa, Artificial Sequence
  Synthetic construct
RQMRAWGQDYQHGGMGYSC

SEQ ID NO: 33 - protein, 20 aa, Artificial Sequence
  Synthetic construct
FWFTLIKTQAKQPARYRRFC

(19) United States
(12) Patent Application Publication
(10) Pub. No.: US 2021/0145972 A1
(43) Pub. Date: May 20, 2021

(54) NANOSTRUCTURE WITH A NUCLEIC ACID SCAFFOLD AND VIRUS-BINDING PEPTIDE MOIETIES

(71) Applicant: FRAUNHOFER-GESELLSCHAFT ZUR FÖRDERUNG DER ANGEWANDTEN FORSCHUNG E.V., München (DE)

(72) Inventors: David Michael Smith, Leipzig (DE); Jessica Sophie Lorenz, Leipzig (DE); Christin Möser, Muldestausee (DE); Jasmin Fertey, Postdam-Golm (DE); Walter Stöcklein, Postdam-Golm (DE); Andreas Herrmann, Berlin (DE); Daniel Lauster, Berlin (DE)

(21) Appl. No.: 16/616,876

(22) PCT Filed: May 25, 2018
(86) PCT No.: PCT/EP2018/063841

§ 371 Date: Nov. 25, 2019

(30) Foreign Application Priority Data
May 26, 2017 (EP) 17173022.9

(51) Int. Cl. A61K 47/54; A61K 38/16; A61P 31/16; A61P 31/14; G01N 33/569

(52) U.S. Cl. CPC A61K 47/549; A61K 38/162; G01N 33/56983; A61P 31/14; A61P 31/16

(57) ABSTRACT

The present invention relates to a nanostructure comprising: a) a nucleic acid scaffold; and b) at least two peptide moieties, wherein the at least two peptide moieties specifically bind to a molecule expressed on the surface of a virus and are attached to the nucleic acid scaffold, wherein the structure of the nucleic acid scaffold is selected from the group of: i) a linear nucleic acid scaffold, wherein the at least two peptide moieties are each attached at or near different ends of the nucleic acid scaffold; and ii) a branched nucleic acid scaffold, wherein the at least two peptide moieties are each attached to a different branch of the scaffold. The invention furthermore relates to the nanostructure of the invention for use as a medicament, and more specifically for use in the treatment of viral infections. The invention also relates to the nanostructure of the invention for the use in diagnostic purposes and in methods of detecting whether a virus is present in a sample.

### BACKGROUND

Antibodies are able to specifically bind to molecules, such as for instance glycoproteins, expressed on the surface of viruses and bacteria. Mostly these molecules are proteins or carbohydrates. Due to their high specificity, antibodies are therefore currently used for diagnosis and treatment of infections arising from pathogens like viruses and bacteria.

Developing and producing such antibodies however is labour intensive and expensive. In addition, antibodies only have limited chemical and thermal stability.

An additional characteristic of natural antibodies is that their structure is highly conserved and it is a labour intensive process for them to be modulated so as to bind a new target with different specificities. Once generated, they are not readily able to be modulated in terms of their binding specificity. New antibodies need to be generated against new targets or mutants, respectively.

In recent years, efforts have therefore been made to produce alternative types of molecules that also specifically bind to a target but can be produced at low cost and with straightforward synthesis at a reasonable scale. One idea would be the implementation of short peptides that are derived from the paratope region of antibodies, maintaining binding features of the source from which they are derived (see for example WO 2016/079250 and EP 3 023 435). However, such short peptides in their monomeric form bind viruses at moderate concentrations in the high micromolar range (Memczak, H. et al. Anti-Hemagglutinin Antibody Derived Lead Peptides for Inhibitors of Influenza Virus Binding. (2016) PLoS One 11).

A multiple peptide presentation on polyglycerol scaffolds targeting the influenza A Virus X31 are able to improve the binding features of monomeric peptides, which was recently shown by Lauster et al. (Angewandte Chemie, 2017, DOI: 10.1002/anie.201702005R1). However, in this work peptides were presented upon a statistically ligand distribution on a flexible polymer scaffold. Further, polymers are known for their broad polydispersity, which is connected to different qualities of independent synthetic batches.

There is therefore a need in the art for a new type of molecule that has the advantages of antibodies and of small peptides but without their respective drawbacks. Such a molecule would have to be able to strongly bind to the surface of a pathogen but be cheap and easy to produce at large scale without variation across different batches. It should also be stable under physiological conditions and be usable for both diagnosis and treatment of infections.

### SUMMARY OF THE INVENTION

The problem was solved by providing nucleic acid based nanostructures, which display virus-binding peptides. Such nanostructures are quick and cheap to produce, because both the peptide moieties as well as the nucleic acid carrier system can be easily synthesised. Nucleic acids can also be easily synthesised and chemically manipulated to contain molecules for attaching peptides or additional features such as fluorescence detection molecules. The nanostructures are also more robust against temperature and pH changes than the bulky and sensitive antibodies and are more flexible in their design because the length and the rigidity as well as the number of branches of the nucleic acid scaffold can easily be adapted to a specific need. Finally, the inventors have surprisingly also found that the nanostructures of the invention can bind to viruses even at low valency, with a much higher affinity than individual short peptides. This realisation implements the stable, cheap and easy production of nanostructures of the invention, which can be used in the diagnosis and treatment of infections.

One embodiment of the invention therefore is a nanostructure comprising:
a) a nucleic acid scaffold; and
b) at least two peptide moieties, wherein the at least two peptide moieties specifically bind to a molecule expressed on the surface of a virus and are attached to the nucleic acid scaffold,
   wherein the nucleic acid scaffold is selected from the group of:
i) a linear nucleic acid scaffold, wherein the at least two peptide moieties are each attached at or near different ends of the nucleic acid scaffold; and
ii) a branched nucleic acid scaffold, wherein the at least two peptide moieties are each attached to a different branch of the scaffold.

In one embodiment, the nanostructure of the invention is for use as a medicament, and more specifically for use in the treatment of a viral infection or in the prophylactic treatment of a viral infection.

One embodiment of the invention is a method of treatment or prophylactic treatment of a viral infection comprising administering to a subject suffering from a viral infection an appropriate amount of a nanostructure according to the invention.

Another embodiment of the invention is the nanostructure of the invention for use in the diagnosis of a viral infection.

A related embodiment is a method of detecting whether a specific virus is present in a sample comprising the steps of:
a. adding to the sample an appropriate amount of a nanostructure of the invention, wherein the peptide moieties of the nanostructure bind under physiological conditions to at least one molecule expressed on the surface of the virus to be detected;
b. incubating the mixture obtained in a. under physiological conditions; and
c. detecting whether the nanostructure has bound to the surface of the virus,
   wherein binding of the nanostructure to the virus is indicative of the presence of the virus in the sample.

### DETAILED DESCRIPTION

The inventions and its different embodiments are disclosed in further details in this section.

One embodiment of the invention is a nanostructure comprising:
a) a nucleic acid scaffold; and
b) at least two peptide moieties, wherein the at least two peptide moieties specifically bind to a molecule expressed on the surface of a virus and are attached to the nucleic acid scaffold,
   wherein the nucleic acid scaffold is selected from the group of:
i) a linear nucleic acid scaffold, wherein the at least two peptide moieties are each attached at or near different ends of the nucleic acid scaffold; and

ii) a branched nucleic acid scaffold, wherein the at least two peptide moieties are each attached to a different branch of the scaffold.

### The Nucleic Acid Scaffold

The nucleic acid scaffold of the nanostructure according to the invention is preferably made of double stranded nucleic acids. A linear nucleic acid scaffold is therefore preferably made of two nucleic acids that anneal to each other under physiological conditions. Preferably, the two nucleic acids anneal to each other over a sufficient length to remain stable in physiological conditions, and more preferably over a segment length comprising at least 12 bases. It should however be understood that the linear scaffold may contain single stranded segments in its interior or on its ends as a necessary means to alter the scaffold's rigidity or add more degrees of freedom to the binding. This single stranded portion of the nucleic acids is preferably 25 or fewer nucleotides in length, more preferably 20 or fewer nucleotides, even more preferably 15 or fewer nucleotides, yet more preferably 10 or fewer nucleotides and most preferably 5 or fewer nucleotides. The skilled person is able to determine when such single stranded stretches are necessary and to design the nucleic acids as required.

When the nucleic acid scaffold is branched, each branch is preferably formed by a double stranded nucleic acid under physiological conditions over a sufficient length to remain stable in physiological conditions, and more preferably over a segment length comprising at least 12 bases. It should however be understood that the portion of the nucleic acids that form part of the branch point may still be single stranded even if the branches of a nucleic acid scaffold are double stranded. Short stretches of single stranded nucleotides may indeed be necessary to accommodate a branch point. It should also be understood that the branched scaffold may contain single stranded segments within the branches or on their ends as a necessary means to alter the branch rigidity or add more degrees of freedom to the binding. This single stranded portion of the nucleic acids at the branch point, within the branches or at the ends of the branches is preferably 25 or fewer nucleotides in length, more preferably 20 or fewer nucleotides, even more preferably 15 or fewer nucleotides, yet more preferably 10 or fewer nucleotides and most preferably 5 or fewer nucleotides. The skilled person is able to determine when such single stranded stretches are necessary and to design the nucleic acids as required.

It will also be clear to the skilled person that some mismatches between two strands can be tolerated without disrupting the annealing between the strands. In a preferred embodiment, the two nucleic acids that anneal to each other to form a nucleic acid scaffold have complementary sequences over the stretch over which they are to anneal (i.e. over the entire length for a linear scaffold and from the branch point to the end of a branch for a branched scaffold) of at least 70%, more preferably 80%, yet more preferably 90%, even more preferably 95%, and most preferably 100%.

In one embodiment of the invention, each of the nucleic acid strands of the scaffold is made of a nucleic acid selected from the group comprising DNA, RNA, LNA, PNA, or any nucleic acids from the class of XNA (xeno nucleic acid—a class of nucleic acids with an unnatural moiety replacing the sugar molecule). The class of XNAs comprises for example CeNA, ANA, FANA, TNA, HNA, LNA, GNA and PNA and the binding affinities of many of them are described for example in Pinheiro, V. B., et al. (Synthetic Genetic Polymers Capable of Heredity and Evolution. (2012) Science, 336, 341-344). In one embodiment, each of the strands is made of a different nucleic acid. In another embodiment, at least one strand comprises several different types of nucleic acids. The stability of a natural nucleic acid hybrid can for example be increased by adding non-natural nucleotides such as LNA and PNA. These offer more stability both because they allow stronger binding between two strands of nucleic acids and/or because they are less likely to be degraded by enzymes. In one embodiment therefore, at least one strand of the nucleic acid comprises at least 2% of a non-natural nucleotide, more preferably at least 5%, even more preferably at least 10%, yet more preferably at least 15% and most preferably at least 20%. These non-natural nucleotides can all be located next to each other in a strand or interspersed among natural nucleotides.

In a preferred embodiment, however, each strand is made of one nucleic acid over its entire length. In a more preferred embodiment, at least two, three, four, five or most preferably all of the nucleic acid strands of the nucleic acid scaffold are made of the same nucleic acid. The nucleic acid strands of the nucleic acid scaffold are preferably made of DNA or RNA. DNA and RNA are particularly preferred nucleic acids because they are easy to manipulate, cheap to produce and extremely well studied. Their properties are therefore well understood and the nucleotide composition of such strands can be easily modulated to obtain the desired properties (length, flexibility, strength of annealing etc. . . . ) of the nucleic acid scaffold. The most preferred nucleic acid is DNA due to its high versatility and stability.

A great advantage of the nucleic acids scaffolds is that once the individual strands have been produced, the scaffold can self-assemble when the mix of nucleic acids is subjected to the appropriate hybridisation conditions. This can be performed before attaching the peptide moieties to the different strands of nucleic acids. Alternatively, the peptides can be attached to the pre-formed nucleic acid scaffold in order to obtain the nanostructures according to the invention.

In one embodiment of the nanostructure, the nucleic acid scaffold is further stabilised by chemical modifications. An example of such a chemical modification is a covalent bond between two individual nucleic acid strands of the scaffold. Such a modification provides the nucleic acid scaffold with higher stability.

In one embodiment, the nanostructure comprises at least one detectable label. A detectable label can be any conjugated molecule that allows the presence of a nanostructure that carries the label to be detected. Suitable detectable labels include fluorescent moieties, radio-labels, biotin and magnetic particles. Many other types of detectable labels are known in the art and the skilled person will be able to determine in each situation which one is the most suitable. A nanostructure that comprises a detectable label is particularly useful for use in diagnosis. The detectable label allows the user to detect whether a nanoparticle is present or absent in the diagnostic assay. The detectable label can be bound to the nucleic acid scaffold or to a peptide of the nanostructure. The detectable label can for example also be an integral part of the peptide in the form for example of a fluorescent amino acid. The label can be bound to the nanoparticle either covalently or non-covalently.

The nucleic acid scaffold of the nanostructure is preferably branched. It therefore preferably comprises more than two ends. It preferably comprises three branches. Since each branch has one end, a nucleic acid scaffold with three branches also comprises three ends. The nanostructure according to the invention is however not limited to a nucleic acid scaffold with three branches. The nucleic acid scaffold may indeed have four, five, six seven, eight, nine or even ten or more branches. Each of these branches can potentially carry at least one peptide moiety that binds to a molecule expressed on the surface of a virus under physiological conditions. Preferably, each of the branches of the nucleic acid scaffold is attached to at least one of such a peptide moiety. The optimal number of branches of the nucleic acid scaffold depends on the application for which the nanostructure is to be used.

In a preferred embodiment of the nanostructure, at least one peptide moiety is attached to essentially each branch of the nucleic acid scaffold.

In the context of this invention, the word “essentially” is to be understood as at least 80%, preferably 85%, more preferably 90%, even more preferably 95%, yet more preferably 98%, and most preferably 100%.

In one embodiment of the nanostructure, each of the branches of the nucleic acid scaffold emanates from the same branch point (from a single junction). In an alternative embodiment, the nucleic acid scaffold comprises more than one branch point and the different branches of the scaffold do not all emanate from the same branch point. This is only possible for nanostructures with more than three branches. When the nanostructure comprises more than three branches, it therefore may comprise one, two, three, four, five, six or more branch points. The number of possible branch points depends on the number of branches of the nanostructure. The possibility of having more than one branch point in the nucleic acid scaffold provides more flexibility in the shape and design of the nanostructures. It indeed allows one to modulate the geometry of the nanostructure and to position the peptide moieties in exactly the required position relative to each other.

A nanostructure with a branched nucleic acid scaffold preferably comprises as many individual nucleic acids as there are branches in the scaffold. This can be expressed as follows: a branched nucleic acid scaffold with n branches preferably comprises n strands of nucleic acids hybridised in such a way as to form the nucleic acid scaffold, wherein n≥3. Each of the individual nucleic acids in such a case is able to hybridise to another nucleic acid of the scaffold essentially from its end to a branch point, and to at least another of the nucleic acids of the scaffold from essentially the branch point to essentially its other end. In the case when the nucleic acid scaffold comprises more than one branch point, the individual nucleic acids that span more than one branch point are hybridised to more than two of the other nucleic acids of the nucleic acid scaffold.

In an alternative embodiment of the nanostructure, the nucleic acid scaffold consists of a single nucleic acid. In this embodiment, at least some of the ends of the nanostructure are in fact a loop to which at least one peptide moiety can be attached. One advantage of a nanostructure with only one nucleic acid strand is that it is even easier and therefore likely cheaper and quicker to produce than a nanostructure that comprises several strands of nucleic acid. Another advantage is that a nucleic acid with fewer ends is more resistant to degradation because most nucleases are exonucleases that degrade nucleic acids from their ends or from breaks in a double strand. Nucleic acids that comprise only one end can for example be produced by using a ligase to ligate together two ends that are in close proximity in a structure. Alternatively, they can be produced by using a single nucleic acid for each nanostructure.

In one embodiment of the nanostructure, each branch has a maximum length, as defined by the distance between the closest branch point to the end of the branch, of 200 nm, preferably 100 nm, more preferably 50 nm, even more preferably 25 nm, yet more preferably 10 nm and most preferably 5 nm. The length of each branch of the nanostructure can also be expressed in length ranges. In one embodiment therefore, each branch of the nanostructure has a length of between 2 nm and 200 nm, preferably between 3 nm and 100 nm, more preferably between 3.5 nm and 50 nm, even more preferably between 4 nm and 25 nm, yet more preferably between 4.5 nm and 10 nm and most preferably 5 nm.

In one embodiment of the nanostructure, the length of each of the branches of the nucleic acid scaffold is selected so as to provide the nanostructure with the optimal binding geometry to bind its target. In one embodiment, different branches of the nucleic acid scaffold are of different lengths. However, in a preferred embodiment, all of the branches of the nucleic acid scaffold are of the same length.

In one embodiment of the nanostructure, the nucleic acid sequences of the nucleic acid scaffold are selected so as to provide rigid arms and flexible branch points.

Rigid branches can be obtained by providing double-stranded branches of a certain length. The rigidity of a double stranded nucleic acid nucleic acid is defined by the material term “persistence length”. If a double-stranded segment is much shorter than the persistence length, then it is effectively rigid. If it is much longer, then it is effectively flexible. For double-stranded DNA, the persistence length is 50 nm. The skilled person will be able to measure or calculate the persistence length of any other nucleic acid used in the nanostructure. In a preferred embodiment therefore, each of the branches has a length of 50% or less of its persistence length, preferably 40% or less, more preferably 35% or less, even more preferably 30% or less, yet more preferably 25% or less and most preferably 20% or less.

### The Peptide Moieties

In one embodiment of the invention, the at least two peptide moieties attached to the nucleic acid scaffold each bind to a molecule expressed on the surface of a virus with a K_{D} of less than 500 μM under physiological conditions. Preferably, this K_{D} is of less than 200 μM, more preferably less than 100 μM, even more preferably less than 50 μM, yet more preferably less than 40 μM, yet more preferably less than 30 μM, and most preferably less than 25 μM.

Methods for determining the equilibrium dissociation constant K_{D} are well known in the art (see for example Memczak, H. et al. Anti-Hemagglutinin Antibody Derived Lead Peptides for Inhibitors of Influenza Virus Binding. (2016) PLoS One 11).

“Physiological conditions” in the context of this document are broadly to be understood as conditions in which normal peptides, proteins and nucleic acids are present in their native form, i.e. they are not denatured. The skilled person is able to determine for each virus and nanostructure of the invention what these conditions are. Physiological conditions are generally mild conditions that can be found inside living organisms or are compatible with living organisms. Broadly, physiological conditions are conditions in which the pH is mild (between 5 and 9, but preferably between 6 and 8), the salt concentration is around 8 grams per litre, and the temperature does not exceed 60° C., but preferably, it does not exceed 50° C. or even more preferably, 40° C.

In one embodiment of the nanostructure, each of the peptide moieties is a peptide of 6 to 150 amino acids, more preferably of 8 to 100 amino acids, even more preferably of 10 to 50 amino acids, yet more preferably of 12 to 25 amino acids, even more preferably of 14 to 20 amino acids and most preferably of 15 amino acids. In any case, the preferred individual peptide moieties are of less than 30, preferably less than 25, more preferably less than 20, and most preferably less than 15 amino acids.

One of the advantages of the present nanostructure is that the peptide components of the structure are small compared for example to antibodies. Each heavy and light chain of a typical antibody comprises over 400 and over 200 amino acids, respectively. The peptide moieties of the nanostructures of the present invention are therefore small in comparison. Indeed, a typical 3-branded structure with 15 nucleotides per branch and one peptide on each branch is approximately 35 kDa. This represents only about one fifth of the size of a typical antibody. In addition to the higher design flexibility this small size offers, nanostructures of the present invention can be produced at a fraction of the cost of antibodies. Producing the two components of the nanostructures, i.e. the nucleic acid strands, especially DNA, and short peptides, is relatively straightforward and cheap. The peptide moieties can indeed preferably be produced by solid-phase peptide synthesis, preferably using the Fmoc- or Boc-strategy. Similarly, DNA strands can be produced in high quality and quantity by solid-phase synthesis. In contrast, antibodies are produced and recovered from animals or cell cultures. This is expensive and such production methods lead to potentially large variations between different batches. In contrast, the production of the nanostructures is much less subject to variation because biological matter is not involved and every production parameter can therefore be much more tightly controlled.

In one embodiment of the invention, the at least two peptide moieties of the nanostructure each bind to different molecules expressed on the surface of the same virus. In a further embodiment, when a nanostructure carries more than two peptide moieties, each of the peptide moieties binds to a different molecule expressed on the surface of a virus. In a preferred embodiment however, the at least two peptide moieties of the nanostructure bind to the same molecule (e.g. an oligomeric protein) expressed on the surface of the target virus. In a more preferred embodiment, all of the peptide moieties of the nanostructure bind to the same molecule expressed on the surface of the target virus. In a particularly preferred embodiment, all of the peptide moieties of a given nanostructure are the same.

The advantage of having two or more peptide moieties that bind to the same molecule on a nanostructure is that the nanostructure may benefit from the cooperative binding effect of the two or more moieties. This allows stronger binding between the nanostructure and the virus, provided the distance between the different peptide moieties allows such binding. The more peptide moieties are attached to the nucleotide scaffold, the higher the potential binding affinity of the nanostructure to the virus, and therefore the higher the potential effect of the nanostructure in the treatment of the infection and the better the potential sensitivity of a method of detection of the virus using the nanostructure. A further advantage of the nanostructures of the invention over antibodies is therefore that they may allow cooperative binding to more than two target molecules.

The mechanism that leads to inhibition of the viruses is the binding of several peptides that are connected through the nanostructures according to the invention. This results in blocked viral receptors that hinders the viruses from binding to or entering cells. Therefore, the peptide moieties of the nanostructures of the invention can be selected to bind to any type of molecule or receptor expressed on the surface of a virus under physiological conditions. However, these peptide moieties preferably each specifically bind to a protein expressed on the surface of a virus, and more preferably to a glycoprotein.

Examples for appropriate virus binding peptides according to the invention are the PeB (SEQ ID NO: 1) and PeBGF (SEQ ID NO: 27) peptides that bind to the influenza A hemagglutinin receptor, the DET4 (SEQ ID NO: 5) and DET2 (SEQ ID NO: 30) peptides that bind to the III domain of the DENV-2 E Protein, the DN57opt (SEQ ID NO: 31), DN81opt (SEQ ID NO: 32) and 1OAN1 (SEQ ID NO: 33) peptides that bind to the II domain of the DENV-2 E Protein, the FluPep 1 (SEQ ID NO: 28) and similar FluPep 2-9 peptides that bind to the influenza hemagglutinin receptor, and the mucroporin-M1 (SEQ ID NO: 29) peptide that binds to the surface of measles, SARS-CoV and influenza viruses. Other examples can include peptides derived by phage display screening against whole virus particles or specific proteins appearing on the surface of the virus, in silico modelling of peptides binding to proteins on the surface of a virus, or proteins derived from the sequences of the active CDR regions of antibodies that are responsible for the binding interaction with proteins on the surface of viruses.

Examples of such glycoproteins expressed on the surface of viruses to which the peptide moieties of the nanostructure can preferably bind are the influenza A hemagglutinin or neuraminidase and the HIV glycoproteins gp41 and gp120. Further examples include the II and III domains on the Dengue virus E Protein or West Nile virus E Protein, the G or F proteins of the respiratory syncytial virus (RSV), the class I fusion protein S2 of the SARS coronavirus, the Class III fusion protein Gb of the human cytomegalovirus, the Class II fusion protein Gn of the Rift Valley virus, and the E1 or E2 envelope glycoproteins if the Hepatitis C virus.

### Design Process For the DNA Structure

The inventive process which is used to design the DNA scaffold, and the subsequent arrangement of the virus-binding peptides, is according to the following:

### Necessary Background Knowledge

There is some background knowledge needed to design the DNA scaffold and to arrange the virus-binding peptides.

A.1 Structural information about the 2D arrangement of the targeted binding sites on the surface of the virus.

Source of information: This knowledge can come from X-ray crystallography, cryo-electron microscopy, atomic force microscopy or other high-resolution methods for structural determination. This information is usually available in publicly available published manuscripts.

By “2D arrangement” according to the invention, a 2D map of the binding sites is meant, when they are sliced by a 2D plane that optimally includes all of the binding sites.

Useful 2D structural information might be as follows:

A.1.1 Overall number (N) and 2D geometry of binding sites on the targeted “protein unit”. This protein unit can be triangular/trimeric (in one embodiment of the invention like influenza hemagglutinin—FIG. 5a), linear/dimeric (in another embodiment of the invention like Dengue E Protein dimer—FIG. 6a), or something else.

A.1.2 Distances between binding sites within a single unit.

A.1.3 Geometric, spatial and orientational relation to other neighboring protein units: an example for Dengue is shown in FIG. 6b. Three dimer protein units are stacked next to each other in a parallel, staggered manner. These triple-dimer stacks also have a specific orientation to other triple-dimer stacks on the surface.

A.2 Structural information about the 3D arrangement of the targeted binding sites on the targeted protein unit.

Source of information: same as in A.1

Useful 3D structural information is as follows:

A.2.1 Are the binding sites accessible within the single plane defined in A.1 (like influenza hemagglutinin—FIG. 5a) or are they occurring around the circumference (like RSV F-Protein—FIG. 7a) of the targeted protein unit?

A.3 Information about the interaction between the virus-binding peptide and the targeted protein structure on the virus.

Caveat: It should be noted that this information is useful for improving the design process, but is not completely necessary. As long as we know that there is an interaction, we can still design the structure and systematically test variations in the design in order to optimize the DNA-peptide structure.

Useful information is as follows:

A.3.1 Binding affinity between peptide and virus: Is the binding interaction “strong” (dissociation constant (Kd) in nM range or lower) or “weak” (above nM range, usually in μM range).

B. Design choices/process for the DNA structure resulting from the background knowledge (A)

B.1 Number of peptides (P) presented by the DNA structure

Reasoning: Having multiple peptides on one structure, which cooperatively bind to the binding sites on the surface of the virus, exploits the multivalence enhancement of binding properties.

B.1.1 If the number of binding sites (N) on the targeted protein unit is at least 3, preferably initially P=N peptides is selected.

B.1.2 If N is 2, the bivalent (2 peptide) binding will likely be too weak, so in one embodiment of the invention the initial selection is based on information in A.1.3.

B.1.3 Depending on the relative arrangement of protein units on the virus surface (A.1.3), also other numbers of peptides might be chosen. For example, if the protein units are closely packed, P>N peptides to increase binding might be chosen.

B.2 Spatial relation between the peptides presented

Reasoning: Ideally, an arrangement of peptides that closely matches the arrangement of binding sites on the virus surface should be achieved. One aim according to the invention is to either match (a) the arrangement of binding sites within a single protein unit (like with influenza HA—FIG. 5b), or (b) the arrangement of binding sites between neighbouring protein units (like with Dengue E-protein—FIG. 6c).

B.2.1 The simplest way to do this is to design a DNA structure consisting of P strands, which is designed in such a way that there are P arms extending outward from a central joint (example—3-arm structure in FIG. 5b). The peptides are then attached at the end of each arm. This is most appropriate when the binding sites are all accessible in a planar arrangement (like the example with influenza—FIG. 5a).

B.2.2 In the branched structure defined in B.2.1, multiple peptides can also be placed onto a single arm, either at the end of the arm, or at internal sites along the arm. This would be appropriate when more complex arrangements are chosen, for the same factors described in B.1.2 and B.1.3.

B.2.3 Similar to B.1.3, also other geometries of DNA structures that are usually occurring may be chosen or designed.

B.3 Lengths of the arms for the branched structures defined in B.2.1

Reasoning: This is simply a tool for precisely controlling the spatial relations.

B.3.1 The simplest way to do this is to vary the number of base pairs in each arm (each dsDNA base-pair=about 0.34 nm)

B.3.2 Another way to control length is to add single-stranded segments (each ssDNA base=about 1 nm) at the central joint, along the arm, or at the end of the arm where the peptides are bound.

B.3.3 The arms can be different lengths relative to each other, depending on the geometry of the binding sites on the virus surface. This is appropriate when binding between neighbouring protein units is the targeted geometry (like the example Dengue—FIG. 6c).

B.4 Flexibility in the spatial relations of the peptides

Reasoning: Based on theories & models of multivalent binding (doi:10.1021/ja1103298), flexibility of the spatial relation between binding sites (in this case but not limited to, peptides) can be used as a control parameter to enhance the binding strength.

Useful background information: dsDNA is approximately 50× stiffer than ssDNA.

B.4.1 Flexibility can be controlled by inserting unpaired ssDNA bases within the central joint

B.4.2 Flexibility can be controlled by inserting mismatches in the base-pairing along the arms

B.4.3 Flexibility can be controlled by appending the dsDNA arm with a ssDNA segment. The peptide is attached to the end of the ssDNA segment.

B.4.4 Flexibility can be controlled by altering the number of strands (e.g. using P-1 strands) or other design choices, so that 1 or more arms are consisting partly or wholly of ssDNA.

B.4.5 Similar to B.1.3 and B.2.3, also other geometries of DNA structures that vary flexibility might be chosen or designed.

B.5 Accounting for 3D structure of protein units

Reasoning: On some protein units on the surface of viruses, for example the F-protein of RSV (FIG. 7a), the binding sites are arranged in a way so that they are not accessible within a single plane. In the case of RSV-F, they are located around the circumference of the protein unit. In these cases, the design of the DNA structure must include features to allow for increased access to all binding sites.

B.5.1 One way to do this is to attach the virus-binding peptides onto flexible ssDNA segments that append the rigid dsDNA arms of a simple branched structure (FIG. 7b).

B.5.2 Similar to B.1.3, B.2.3 and B.4.5, also choose/design other geometries of DNA structures that account for the 3D structure which are possible may be chosen or designed.

The spatial arrangement of the anti virus binding peptides over the nucleic scaffold is crucial for the inhibitory effect of the nanostructures according to the invention.

In one embodiment of the nanostructure, each of the peptide moieties attached to the nucleic acid scaffold binds to a molecule expressed on the surface of a virus selected from the groups of orthomyxoviridae (such as influenza A), filoviridae (such as the Ebola virus), retroviridae (such as HIV), coronaviridae (such as the SARS-coronavirus), togaviridae (such as the alphavirus), flaviviridae (such as Dengue virus, West Nile virus, Zika virus, Yellow fever virus), and pneumoviridae (such as the human respiratory syncytial virus). Preferably, all of the peptide moieties of a nanostructure bind to a protein expressed on the surface of any one of the above viruses. More preferably, each of the peptide moieties of a nanostructure binds to proteins on the surface of the same virus, and even more preferably, they all bind to the same protein.

In a preferred embodiment of the nanostructure, at least one, but preferably all, of the peptide moieties attached to the nucleic acid scaffold bind to a viral fusion protein. Preferably, the viral fusion protein is a viral fusion protein of class I or class II. The viral fusion proteins of class I and II are particularly good targets because they are present on the surface of the viruses as homotrimers (class I) or homodimers (class II). This allows di- or multivalent binders such as the nanostructures according to the invention to target the viruses more effectively because several targets are present in close proximity, which allows cooperative binding of the different peptide moieties. Preferably, the number of peptide moieties attached to a nanostructure according to the invention corresponds to the number of target proteins that are present in a homopolymer on the target virus. For example, if the influenza A hemagglutinin is to be targeted by the nanostructure, the number of hemagglutinin-binding peptide moieties carried by each nanostructure is preferably three, because the hemagglutinin is present on the surface of the virus as a homotrimer.

In a preferred embodiment of the nanostructure, the at least one, or more preferably all, of the peptide moieties of the nanostructure bind to a viral fusion protein of class I. In an even more preferred embodiment, all of the peptide moieties bind to the same viral fusion protein of class I.

Viral fusion proteins are particularly preferred targets because the inventors have surprisingly found that the multivalent binding of the nanostructures to a virus can prevent the virus from interacting, and therefore fusing, with the host cell. Binding of the nanostructures of the invention to a virus will therefore likely prevent the virus from infecting its target cell.

With some viruses, there are separate proteins for binding to host cells and fusion with the host cell. For example, with RSV, the F protein is the fusion protein, and the G protein is for recognition/binding. In such cases, either a fusion protein, a recognition/binding protein or both can be targeted by the nanostructure.

In one embodiment of the nanostructure, at least one of the peptide moieties is derived from the complementarity determining region of an existing antibody. The way to do this is known in the art. Memczak, H. et al. (Anti-Hemagglutinin Antibody Derived Lead Peptides for Inhibitors of Influenza Virus Binding. (2016) PLoS One 11) for example discloses the design of peptide moieties that bind to the influenza A hemagglutinin protein based on the HC19 antibody. In another embodiment, at least one of the peptide moieties is derived from the phage display method. This method allows to screen peptides for their affinity to a particular target. It therefore allows the identification of new peptides that have the required binding affinity against a target of interest. Such peptides can then be used in the nanostructures of the invention.

In a preferred embodiment of the nanostructure, at least one of the peptide moieties binds to influenza A hemagglutinin. More preferably, at least two, or even more preferably all, of the peptide moieties of the nanostructure bind to the influenza A hemagglutinin. One suitable peptide for this purpose is for example the PeB peptide (ARDFYDYDVFYYAMD—SEQ ID NO: 1), or a peptide that comprises the PeB peptide. The dissociation constant K_{D} of this peptide with hemagglutinin has indeed been shown by Memczak, H. et al. (Anti-Hemagglutinin Antibody Derived Lead Peptides for Inhibitors of Influenza Virus Binding. (2016) PLoS One 11) to be 56.8 μM. Preferably, each of the peptide moieties of the nanostructure comprises, or even more preferably consists of, the peptide PeB (SEQ ID NO: 1).

In another preferred embodiment of the nanostructure, at least one of the peptide moieties binds to Dengue virus E-Protein. More preferably, at least two, or even more preferably all, of the peptide moieties of the nanostructure bind to Dengue virus E-Protein. One suitable peptide for this purpose is the DET4 peptide (AGVKDGKLDF—SEQ ID NO: 5), or a peptide that comprises the DET4 peptide.

The entry of virus entry into host cells was indeed shown by Alhoot et al. (Inhibition of dengue virus entry into target cells using synthetic antiviral peptides. (2013) Int. J. Med. Sci 10) to be inhibited by 50% (IC₅₀) when 35 μM of the peptide was applied. Preferably, each of the peptide moieties of the nanostructure comprises, or even more preferably consists of, the peptide DET4 (SEQ ID NO: 5).

In a preferred embodiment of the invention, the peptide moieties are attached to the nucleic acid scaffold by way of a covalent link. The skilled person knows how to covalently attach a peptide moiety to a nucleic acid. This can for example be performed by Click chemistry, in which one of the two molecules to be linked carries an azide group and the other a dibenzocyclooctyne (DBCO) moiety. The two molecules are then linked together by cycloaddition.

In a preferred embodiment of the nanostructure, the nucleic acid scaffold is branched. Preferably in such a case, a peptide moiety is attached at or near the end of each of the branches of the nucleic acid scaffold. More preferably, each of the peptide moieties is attached at the end of each of the branches. The peptide moieties can be attached to the nucleic acids of the nucleic acid scaffold directly or via a linker. The linker can optionally be cleavable.

A peptide moiety that is attached “at or near” an end or a branch of a nucleic acid scaffold is to be understood in the context of this invention as a peptide moiety attached to a nucleotide that is located at most 20 nucleotides from the end of the double stranded segment of the nucleic acid strand. More preferably, the peptide moiety is bound to a nucleotide located at most 15 nucleotides from the end of the nucleic acid strand, more preferably at most 10 nucleotides, even more preferably at most 5 nucleotides, yet more preferably at most 2 nucleotides. In the most preferred embodiment, the peptide moiety is attached to the last nucleotide of the double stranded segment of the nucleic acid scaffold end or branch. When the nucleic acid scaffold is made of double stranded nucleic acids, a peptide moiety can be attached to the terminal nucleotides of both of the nucleic acid strands. Preferably however, the peptide moiety is bound to the last nucleotide of only one strand of the double stranded end or branch.

In one embodiment of the nanostructure, more than one peptide is attached at or near the end of at least one of the nucleic acid scaffold ends. Preferably in this case, at least two, three four, five or six peptides are attached at or near the end of a single nucleic acid scaffold end. All the peptide attached to a single end can be different peptides, however it is preferred that all of the peptides attached at or near a single end are the same peptide. The attachment of more than one peptide at or near the end of one or several of the branches of the nucleic acid scaffold provides increased avidity for the target.

There are some designs however in which it is advantageous to have a peptide on both ends of a single DNA strand. This is for example the case in a 3-branch structure with 2 of the branches completely flexible and single-stranded that is made using only 2 strands. In such an embodiment, at least one of the nucleic acids of the nanostructure carries a peptide on both ends in order for the nanostructure to comprise a peptide on each of the branch ends. In one embodiment of the nanostructure therefore, one or more of the nucleic acid strands of the nanostructure carry a peptide on each of the 5′ and 3′ ends.

In one embodiment of the nanostructure of the invention, the distance between the individual moieties attached to the scaffold is, under physiological conditions, between 3 nm and 100 nm, preferably between 4 nm and 80 nm, more preferably between 5 nm and 60 nm, even more preferably between 6 nm and 40 nm, yet more preferably between 7 and 20 nm, yet more preferably between 8 and 10 nm and most preferably 9 nm.

In one embodiment, the distance between the binding moieties is selected so as to allow optimal binding to the target virus. This optimal distance depends on the conformation and the distribution of the target molecules on the virus. The optimal distance between the peptide moieties can be achieved by adjusting the length of the branches, their rigidity, and the flexibility of the branch point(s) of the nucleic acid scaffold. In a preferred embodiment, these properties are therefore adjusted to optimise the binding of the nanostructure to its target virus.

### Further Embodiments

All of the embodiments of the nanostructure described above also apply to the further embodiments of this section.

A further embodiment of the invention is a composition comprising nanostructures according to the invention in which essentially each branch of each nucleic acid scaffold is attached to a virus-binding peptide moiety. The word essentially in this context is to account for the fact that the attachment of a peptide moiety to a nucleic acid may not be 100% efficient. As a result, a certain number of nucleic acid scaffolds in a composition will not carry a peptide moiety on each of their branches. Preferably however, at least 60% of the nucleic acid scaffolds of the composition carry a peptide moiety on each of their branches, more preferably at least 80%, even more preferably at least 90%, yet more preferably at least 95% and most preferably 100% of them do.

In one embodiment, the nanostructures of the invention are for use as a medicament. More specifically, the nanostructures of the invention are for use in the treatment of a viral infection. As will be clear to the skilled person, a nanostructure with peptides for use in the treatment of an infection with a specific virus should carry at least one peptide moiety that binds to at least one molecule expressed on the surface of the target virus. Preferably, all of the peptide moieties of the nanostructure bind to a molecule expressed on the surface of the target virus. In a preferred embodiment, the viral infection is an infection with a virus selected from the group comprising influenza A, the togavirus, the human respiratory syncytial virus, the Dengue virus, the West Nile virus, the Zika virus and the Yellow fever virus.

The nanostructures of the invention can also be used for prophylactic treatment, to prevent a viral infection. As a consequence, one embodiment of the invention is the nanostructures of the invention for use in the prophylactic treatment of a viral infection.

The nanostructures of the invention can likely be used for the (prophylactic) treatment of viral infections due to the ability of the nanostructures to strongly bind to their target viruses. The DNA-PeB₃ nanostructure described more in detail in the example below is for example able to inhibit binding of the influenza A virus to red blood cells. Similarly, the DNA-DET4 nanostructure described in more detail in the supporting example below inhibits the entry of Dengue into Vero E6 cells. It is highly unlikely that these viruses would be able to infect cells that it cannot bind to and/or enter. It is highly unlikely that the virus would be able to infect cells that it cannot bind to.

One embodiment of the invention is the nanostructure according to the invention for use in the treatment of a viral infection or for the prevention of a viral infection.

One embodiment of the invention is the nanostructure according to the invention for use in the in vivo diagnosis of a viral infection. The diagnosis of the viral infection is performed by detecting the presence of a virus in the body.

One embodiment of the invention is the nanostructure according to the invention for use in the diagnosis of a viral infection. The diagnosis of the viral infection is performed by detecting the presence of a virus in a sample that has been taken from a subject using a nanostructure according to the invention.

A sample that has been taken from a subject can for example be any body fluid such as any fluid that is excreted or secreted from the body. Typically, the body fluid is blood, serum, saliva, tear fluid, lymphatic fluid, urine or sweat. Such a sample can also be a tissue sample, for instance a mucosa sample, and more specifically an oral mucosa sample. The skilled person will be able to determine which sample is the most appropriate in each case.

One embodiment of the invention is also a method of detecting whether a specific virus is present in a sample comprising the steps of:
a. adding to the sample an appropriate amount of a nanostructure of the invention, wherein the peptide moieties of the nanostructure bind under physiological conditions to at least one molecule expressed on the surface of the virus to be detected;
b. incubating the mixture obtained in a. under physiological conditions; and
c. detecting whether the nanostructure has bound to the surface of the virus,
   wherein binding of the nanostructure to the virus is indicative of the presence of the virus in the sample. The sample in such a method can be a sample that has been taken from a subject. This method can therefore be used for the diagnosis of an infection with a virus.

The detection of whether nanostructures have bound to the surface of a virus can be carried out for example by detecting the nucleic acid of the nanostructure on the surface of the virus by microscale thermophoresis (MST) or some other appropriate means. Alternatively, the detection can be performed indirectly by a test such as the hemagglutination inhibition assay (HAI) in which the antibodies are replaced by the nanostructures according to the invention.

Other assays that can be used are a label free detection assays such as surface plasmon resonance (SPR) or quartz crystal microbalance (QCM); surface acoustic wave (SAW), where virus binding to a surface modified with the nanostructure is directly detected; electrochemical detection, e.g. impedance changes when a virus binds to the nanostructure localised between interdigitated electrodes; a reporter (HRP, fluorophore) conjugated nucleic acid assay; Enzyme-linked Immunosorbent Assay (ELISA); fluorescence quenching, if binding of the virus to dye-labelled nanostructures affects fluorescence; or a strip-based immunodiagnostic test (e.g. on paper). The skilled person will be able to determine in each case which is the most appropriate detection method. The most preferred assay where possible is HAI assay.

Also preferred is an infection inhibition assay. The infection inhibition assay using MDCKII cells and the MTT Test (or MTS). Inhibitor (peptide-conjugates DNA nanostructure) and virus are pre-incubated for 10-30 min at room temperature and added to cells (MDCKII for influenza, Vero E6 for Dengue). Viral infection of host cells can be tested after 24-72 h by either crystal violet staining or MTT/MTS reagents. Infected cells will slow down their metabolism and/or die after a certain time.

A further embodiment of the invention is a method of treatment of a viral infection comprising administering to a subject suffering from a viral infection an appropriate amount of a nanostructure according to the invention. It will be clear to the skilled person that in order for the treatment to work, the peptide moieties of the nanostructure used in the method of treatment have to specifically bind to a molecule expressed on the surface of the virus to be targeted.

A related embodiment of the invention is a method of preventing a viral infection comprising administering to a subject suffering from a viral infection an appropriate amount of a nanostructure according to the invention.

### Nanostructures That Bind to Bacteria

The nanostructures of the invention are not restricted to nanostructures that bind to the surface of viruses. The nanostructures of the invention can indeed carry peptide moieties that bind to molecules expressed on the surface of pathogens other than viruses. If the peptides bind to targets expressed on the surface of bacteria for example, the nanostructures can be used for the (prophylactic) treatment and diagnosis of bacteria instead of viruses. Accordingly, nanostructures with peptides that bind to bacterial targets instead of viral targets are also part of the invention, as well as their uses in treatment and diagnosis.

One embodiment of the invention therefore is a nanostructure comprising:
a) a nucleic acid scaffold; and
b) at least two peptide moieties, wherein the at least two peptide moieties specifically bind to a molecule expressed on the surface of a bacteria and are attached to the nucleic acid scaffold,
   wherein the nucleic acid scaffold is selected from the group of:
i) a linear nucleic acid scaffold, wherein the at least two peptide moieties are each attached at or near different ends of the nucleic acid scaffold; and
ii) a branched nucleic acid scaffold, wherein the at least two peptide moieties are each attached to a different branch of the scaffold.

In one embodiment, the nanostructure of the invention is for use as a medicament, and more specifically for use in the treatment of a bacterial infection or in the prophylactic treatment of a bacterial infection.

One embodiment of the invention is a method of treatment or prophylactic treatment of a bacterial infection comprising administering to a subject suffering from a bacterial infection an appropriate amount of a nanostructure according to the invention.

Another embodiment of the invention is the nanostructure of the invention for use in the diagnosis of a bacterial infection.

A related embodiment is a method of detecting whether a specific bacterium is present in a sample comprising the steps of:
a. adding to the sample an appropriate amount of a nanostructure of the invention, wherein the peptide moieties of the nanostructure bind under physiological conditions to at least one molecule expressed on the surface of the bacteria to be detected;
b. incubating the mixture obtained in a. under physiological conditions; and
c. detecting whether the nanostructure has bound to the surface of the bacteria,
   wherein binding of the nanostructure to the bacteria is indicative of the presence of the bacteria in the sample.

All the embodiments relating to the nucleic acid scaffold disclosed in this document equally apply to nanoparticles that target viruses and bacteria.

In one embodiment of the invention, the at least two peptide moieties attached to the nucleic acid scaffold each bind to a molecule expressed on the surface of a bacteria with a K_{D} of less than 500 μM under physiological conditions. Preferably, this K_{D} is of less than 200 μM, more preferably less than 100 μM, even more preferably less than 50 μM, yet more preferably less than 40 μM, yet more preferably less than 30 μM, and most preferably less than 25 μM.

In one embodiment of the nanostructure, each of the peptide moieties is a peptide of 6 to 150 amino acids, more preferably of 8 to 100 amino acids, even more preferably of 10 to 50 amino acids, yet more preferably of 12 to 25 amino acids, even more preferably of 14 to 20 amino acids and most preferably of 15 amino acids. In any case, the preferred individual peptide moieties are of less than 30, preferably less than 25, more preferably less than 20, and most preferably less than 15 amino acids.

In one embodiment of the invention, the at least two peptide moieties of the nanostructure each bind to different molecules expressed on the surface of the same bacteria. In a further embodiment, when a nanostructure carries more than two peptide moieties, each of the peptide moieties binds to a different molecule expressed on the surface of a bacterium. In a preferred embodiment however, the at least two peptide moieties of the nanostructure bind to the same molecule (e.g. an oligomeric protein) expressed on the surface of the target bacteria. In a more preferred embodiment, all of the peptide moieties of the nanostructure bind to the same molecule expressed on the surface of the target bacteria. In a particularly preferred embodiment, all of the peptide moieties of a given nanostructure are the same.

The peptide moieties of the nanostructures of the invention can be selected to bind to any type of molecule expressed on the surface of a bacterium under physiological conditions. However, these peptide moieties preferably each specifically bind to a protein expressed on the surface of a bacterium, and more preferably to a glycoprotein.

In one embodiment of the nanostructure, at least one of the peptide moieties is derived from the complementarity determining region of an existing antibody. The way to do this is known in the art. Memczak, H. et al. (Anti-Hemagglutinin Antibody Derived Lead Peptides for Inhibitors of Influenza Virus Binding. (2016) PLoS One 11) for example discloses the design of peptide moieties that bind to the influenza A hemagglutinin protein based on the HC19 antibody. In another embodiment, at least one of the peptide moieties is derived from the phage display method. This method allows one to screen peptides for their affinity to a particular target. It therefore allows the identification of new peptides that have the required binding affinity against a target of interest. Such peptides can then be used in the nanostructures of the invention.

In a preferred embodiment of the invention, the peptide moieties are attached to the nucleic acid scaffold by way of a covalent link. The skilled person knows how to covalently attach a peptide moiety to a nucleic acid. This can for example be performed by Click chemistry, in which one of the two molecules to be linked carries an azide group and the other a dibenzocyclooctyne (DBCO) moiety. The two molecules are then linked together by cycloaddition.

In a preferred embodiment of the nanostructure, the nucleic acid scaffold is branched. Preferably in such a case, a peptide moiety is attached at or near the end of each of the branches of the nucleic acid scaffold. More preferably, each of the peptide moieties is attached at the end of each of the branches. The peptide moieties can be attached to the nucleic acids of the nucleic acid scaffold directly or via a linker. The linker can optionally be cleavable.

In one embodiment of the nanostructure, more than one peptide is attached at or near the end of at least one of the nucleic acid scaffold ends. Preferably in this case, at least two, three four, five or six peptides are attached at or near the end of a single nucleic acid scaffold end. All the peptide attached to a single end can be different peptides, however it is preferred that all of the peptides attached at or near a single end are the same peptide. The attachment of more than one peptide at or near the end of one or several of the branches of the nucleic acid scaffold provides increased avidity for the target.

There are some designs however in which it is advantageous to have a peptide on both ends of a single DNA strand. This is for example the case in a 3-branch structure with 2 of the branches completely flexible and single-stranded that is made using only 2 strands. In such an embodiment, at least one of the nucleic acids of the nanostructure carries a peptide on both ends in order for the nanostructure to comprise a peptide on each of the branch ends. In one embodiment of the nanostructure therefore, one or more of the nucleic acid strands of the nanostructure carry a peptide on each of the 5′ and 3′ ends.

In one embodiment of the nanostructure of the invention, the distance between the individual moieties attached to the scaffold is, under physiological conditions, between 3 nm and 100 nm, preferably between 4 nm and 80 nm, more preferably between 5 nm and 60 nm, even more preferably between 6 nm and 40 nm, yet more preferably between 7 and 20 nm, yet more preferably between 8 and 10 nm and most preferably 9 nm.

In one embodiment, the distance between the binding moieties is selected so as to allow optimal binding to the target bacteria. This optimal distance depends on the conformation and the distribution of the target molecules on the bacteria. The optimal distance between the peptide moieties can be achieved by adjusting the length of the branches, their rigidity, and the flexibility of the branch point(s) of the nucleic acid scaffold. In a preferred embodiment, these properties are therefore adjusted to optimise the binding of the nanostructure to its target bacteria.

The nanostructures that are specific for bacterial targets preferably comprise at least two peptide moieties that are capable of binding to target molecules expressed on the surface of Enteroaggregative E. coli (EAggEC; food poisoning), Helicobacter pylori (gastroduodenal ulcers and gastric cancer) and Bordetella pertussis (whooping cough (pertussis)).

In one embodiment, the nanostructure comprises at least one peptide moiety that specifically binds to bacterial HA under physiological conditions. In a preferred embodiment, all of the peptide moieties of the nanostructure bind to the bacterial HA. The nanostructures of the invention that are specific for bacteria are however not limited to nanostructures that comprise such binding moieties. They can comprise peptide moieties that bind to any other target expressed on the surface of the bacteria that are to be detected or treated.

A further embodiment of the invention is a composition comprising nanostructures according to the invention in which essentially each branch of each nucleic acid scaffold is attached to a bacteria-binding peptide moiety. The word essentially in this context is to account for the fact that the attachment of a peptide moiety to a nucleic acid may not be 100% efficient. As a result, a certain number of nucleic acid scaffolds in a composition will not carry a peptide moiety on each of their branches. Preferably however, at least 60% of the nucleic acid scaffolds of the composition carry a peptide moiety on each of their branches, more preferably at least 80%, even more preferably at least 90%, yet more preferably at least 95% and most preferably 100% of them do.

In one embodiment, the nanostructures of the invention are for use as a medicament. More specifically, the nanostructures of the invention are for use in the treatment of a bacterial infection.

As will be clear to the skilled person, a nanostructure with peptides for use in the treatment of an infection with a specific bacterium should carry at least one peptide moiety that binds to at least one molecule expressed on the surface of the target bacteria. Preferably, all of the peptide moieties of the nanostructure bind to a molecule expressed on the surface of the target bacteria.

The nanostructures of the invention can also be used for prophylactic treatment, to prevent a bacterial infection. As a consequence, one embodiment of the invention is the nanostructures of the invention for use in the prophylactic treatment of a bacterial infection.

The nanostructures of the invention can likely be used for the (prophylactic) treatment of bacterial infections due to the ability of the nanostructures to strongly bind to their target bacteria.

One embodiment of the invention is the nanostructure according to the invention for use in the treatment of a bacterial infection or for the prevention of a bacterial infection.

One embodiment of the invention is the nanostructure according to the invention for use in the in vivo diagnosis of a bacterial infection. The diagnosis of the bacterial infection is performed by detecting the presence of a type of bacteria in the body.

One embodiment of the invention is the nanostructure according to the invention for use in the diagnosis of a bacterial infection. The diagnosis of the bacterial infection is performed by detecting the presence of a type of bacteria in a sample that has been taken from a subject using a nanostructure according to the invention.

A sample that has been taken from a subject can for example be any body fluid such as any fluid that is excreted or secreted from the body. Typically, the body fluid is blood, serum, saliva, tear fluid, lymphatic fluid, urine or sweat. Such a sample can also be a tissue sample, for instance a mucosa sample, and more specifically an oral mucosa sample. The skilled person will be able to determine which sample is the most appropriate in each case.

One embodiment of the invention is also a method of detecting whether a specific type of bacteria is present in a sample comprising the steps of:
a. adding to the sample an appropriate amount of a nanostructure of the invention, wherein the peptide moieties of the nanostructure bind under physiological conditions to at least one molecule expressed on the surface of the bacteria to be detected;
b. incubating the mixture obtained in a. under physiological conditions; and
c. detecting whether the nanostructure has bound to the surface of the bacteria,
   wherein binding of the nanostructure to the bacteria is indicative of the presence of the bacteria in the sample. The sample in such a method can be a sample that has been taken from a subject. This method can therefore be used for the diagnosis of an infection with a specific type of bacteria.

The detection of whether nanostructures have bound to the surface of a bacteria can be carried out for example by detecting the nucleic acid of the nanostructure on the surface of the bacteria by microscale thermophoresis (MST) or some other appropriate means. Alternatively, the detection can be performed indirectly by a test such as the hemagglutination inhibition assay (HAI) in which the antibodies are replaced by the nanostructures according to the invention.

Other assays that can be used are a label free detection assays such as surface plasmon resonance (SPR) or quartz crystal microbalance (QCM); surface acoustic wave (SAW), where bacteria binding to a surface modified with the nanostructure is directly detected; electrochemical detection, e.g. impedance changes when a bacteria binds to the nanostructure localised between interdigitated electrodes; a reporter (HRP, fluorophore) conjugated nucleic acid assay; Enzyme-linked Immunosorbent Assay (ELISA); fluorescence quenching, if binding of the bacteria to dye-labelled nanostructures affects fluorescence; or a strip-based immunodiagnostic test (e.g. on paper). The skilled person will be able to determine in each case which is the most appropriate detection method. The most preferred assay where possible is HAI assay.

A further embodiment of the invention is a method of treatment of a bacterial infection comprising administering to a subject suffering from a bacterial infection an appropriate amount of a nanostructure according to the invention. It will be clear to the skilled person that in order for the treatment to work, the peptide moieties of the nanostructure used in the method of treatment have to specifically bind to a molecule expressed on the surface of the bacteria to be targeted.

A related embodiment of the invention is a method of preventing a bacterial infection comprising administering to a subject suffering from a bacterial infection an appropriate amount of a nanostructure according to the invention.

### EXAMPLES

The examples presented here are particular embodiments of the invention. The invention is however not limited to these particular embodiments.

### Example 1

### DNA Scaffold For Nanostructure

A DNA scaffold for a nanostructure of the invention can for example be made by a method with the following steps:
1. synthesising the three DNA strands:

SEQ ID NO: 2 strand
5′-ACTATCTTTGGTCTATTATCTTGAGTCATC-3′
SEQ ID NO: 3 strand
5′-TAGTTGTGTGTGTGTTAGACCAAAGATAGT-3′
SEQ ID NO: 4 strand
5′-GATGACTCAAGATAAACACACACACAACTA-3′

or
synthesising the three DNA strands:

SEQ ID NO: 6 strand
5′-ACGAT CTTTG TTCTA CTGAT GCCTG ACTGA TCCAT GTTAT
ATTGA GTGAT GTACA AATCG GCGTA GTGAA GC-3′
SEQ ID NO: 7 strand
5′-TAGTT GTGTG TGTGT CATGG ATCAG TCAGG CATCA GTAGA
ACAAA GATCG T-3′
SEQ ID NO: 8 strand
5′-GCTTC ACTAC GCCGA TTTGT ACATC ACTCA ATATA AACAC
ACACA CAACT A-3′

2. combining the three strands under conditions that allow self-assembly of the nanostructure scaffold.

The peptide moieties can be attached to the nucleic acid strands prior to assembly or after assembly of the scaffold (see example 2). The peptide moieties are preferably attached either to the 5′ or the 3′ end.

### Example 2

### DNA-PeB₃ Nanostructures

One of the possible nanostructures of the invention is a nanostructure with a DNA nucleic acid scaffold made of three DNA strands (such as the one of example 1) and three PeB peptide moieties, each attached to the end of one of the DNA strands. The peptide moieties are preferably attached to the 5′ end, but attachment to the 3′ end is also possible. Such a nanostructure is schematically represented in FIG. 1A.

The peptide moieties were attached to the nucleic acids of the nanostructure as follows. The peptides are each derivatised with an azide group and each DNA strand of the nucleic acid scaffold with a DBCO linker. Three peptide moieties are then coupled to each nucleic acid scaffold by copper-free Click chemistry. The nanostructures are then purified via size-exclusion chromatography and then run on a native polyacrylamide gel and stained with SYBR® Gold Nucleic Acid Gel Stain (FIG. 1B).

### Example 3

### Binding of the DNA-PeB₃ Nanostructure to its Target

The binding of the DNA-PeB₃ nanostructures to influenza A viruses was tested by the hemagglutination inhibition assay (HAI). The principle on which this assay is based is shown in FIG. 3. Briefly, red blood cells (RBCs) sediment at the bottom of the V or U bottom well in a reaction mixture that comprises only red blood cells RBCs (see FIG. 3, line A). When a virus that expresses hemagglutinin on its surface is also present in the sample, the red blood cells are unable to sediment because they are attached to each other by bridging viruses (FIG. 3, line B). When an anti-hemagglutinin antibody is added to the sample, the RBCs once again sediment at the bottom of the well (FIG. 3, line C). This is because the hemagglutinins on the surface of the virus are bound by antibodies and can therefore no longer interact with the RBCs.

The same HAI can be used to test whether PeB monomers and DNA-PeB₃ nanostructures can bind to influenza A viruses. The only difference is that the antibodies of the HAI as shown in FIG. 3 are replaced by PeB monomers or DNA-PeB₃.

The results of this experiment are presented in FIG. 4A. It can be seen there that the inhibition of the viruses is an impressive and unexpected 263 times more effective with the DNA-PeB₃ nanostructures than with PeB monomers.

A further experiment that was carried out was to directly measure the binding of DNA-PeB nanostructures with different numbers of PeB peptide moieties to the influenza virus. This measurement was performed by microscale thermophoresis, which measures the changes in temperature-dependent movement of molecules by binding of a ligand. Here the ligand was bromelain treated hemagglutinin (BHA) which is soluble and maintains the trimeric structure. This measurement was performed using three-branched DNA nucleic acid scaffolds that carry zero, one, two or three PeB peptide moieties (FIG. 4B). The striking result of this experiment is that only the nanostructure with the three PeB peptide moieties is able to efficiently bind to the BHA. This is likely due to the fact that the cooperative binding of three moieties is necessary to overcome the weak binding of single peptides to the virus.

### Example 4

### Screening For the Binding and Inhibition of Influenza A Virus Particles With PeB-Conjugated DNA Nanostructures

In this case, a collection of different DNA scaffolds that exploit the design choices (e.g. flexibility, size, geometrical arrangement) were tested for their binding to influenza A particles, and for their ability to impede the entry and subsequent infection of the viruses in host cells. A total of 7 different structures (N, U, V, W, X, Y, Z) were tested. Structure N is the same structure used above in examples 1-3. All structures are shown in FIG. 9, sequences are given in the sequence listing SEQ ID NOs 2-4 and 6-22, and include design considerations described earlier in the section titled “Design process for the DNA structure”. An additional DNA scaffold was used for the confirmation of binding by surface plasmon resonance (SPR). This mimics the “N” structure, however with the addition of a fourth arm, which bears a biotin molecule at the end for binding to a streptavidin-coated SPR chip. Sequences for this structure correspond to SEQ ID NOs: 2, 3, and 4.

The peptides are each derivatised with an azide group and each DNA strand of the nucleic acid scaffold with a DBCO linker. Three peptide moieties are then coupled to each nucleic acid scaffold by copper-free Click chemistry. The nanostructures are then purified via size-exclusion chromatography and then run on a native polyacrylamide gel and stained with SYBR® Gold Nucleic Acid Gel Stain, in the same way as was carried out for the DNA scaffolds in Example 2.

The results of those experiments are shown in the FIGS. 8 to 14. The inhibition of the influence virus by different DNA scaffolds together with PeB is shown. It can be seen from SPR data in FIG. 8 that the binding of virus particles is increased when more peptides are presented on the DNA scaffold. From microscale thermophoresis data in FIG. 10, a dose-dependent increase in binding of the DNA-peptide structure to virus particles is seen for structures W and Z when they are carrying three peptides. No binding to the virus particles is seen when they are not carrying any peptides. From hemagglutinin inhibition assays in FIGS. 11-13, a concentration-dependent increase in binding can be seen for structures where 2 or more peptides can bind cooperatively to the hemagglutinin protein on the surface of the virus particle (N, U, V, W, X, Z), and no detectable binding is seen for the structure where cooperative binding of two or more peptides is not favored (Y). For direct detection of infection through observation of cytopathic effect, a clear inhibition of influenza A infection of MDCKII cells is seen for structure N with 3 peptides (FIG. 14C), while all other conditions (virus treatment—FIG. 14A; N without peptides—FIG. 14D; PeB peptide by itself at 500 μM—FIG. 14E), a more significant cytopathic effect, and therefore infection by viruses is seen.

### Example 5

### DNA-Dengue Structures

Another possible nanostructure of the invention is a nanostructure with a DNA nucleic acid scaffold made of three DNA strands (such as the one of example 1) and three DET4 peptide moieties, each attached to the end of one of the DNA strands. The peptide moieties are preferably attached to the 5′ end, but attachment to the 3′ end is also possible. Such a nanostructure is schematically represented in structure “Y” in FIG. 9. A schematic of the binding of this structure to the lattice of E-Proteins on the surface of the Dengue virus is shown in FIG. 6c.

The peptide moieties were attached to the nucleic acids of the nanostructure as follows. The peptides are each derivatised with an azide group and each DNA strand of the nucleic acid scaffold with a DBCO linker. Three peptide moieties are then coupled to each nucleic acid scaffold by copper-free Click chemistry. The nanostructures are then purified via size-exclusion chromatography and then run on a native polyacrylamide gel and stained with SYBR® Gold Nucleic Acid Gel Stain.

### Example 6

### Inhibition of Dengue Infection by DNA Structures

The inhibition of Dengue virus 2 by DET4 peptides and DET4 coupled to DNA nanostructures was investigated by focus formation assay. Target cells were seeded in a 96-well plate the day before infection. In a separate 96 well plate 200-500 virus particles per well were incubated with or without DET4 monomers and DET4-coupled DNA Trimers in different concentrations for 2 hours followed by transfer of the mixtures to confluent target cell monolayers and incubation for 1 h at 37° C.

Subsequently fresh DMEM/2% (v/v) FBS containing 1.6% (w/v) Avicell was added to each well, covering the surface with microcrystalline cellulose to avoid spreading of the virus in the medium and enhancing formation of virus foci. Infected cells were incubated for 4 days followed by washing with PBS and fixing with 70% ethanol. Cells were rinsed with phosphate buffered saline, pH 7.4 (PBS) prior to immunostaining. Virus foci were detected using an Anti-flavivirus mouse monoclonal antibody that recognizes Dengue viruses, followed by horseradish peroxidase-conjugated goat anti-mouse immunoglobulin, and developed using TMB chromogen substrate. After washing, Virus foci were visible in the wells as blue spots that were counted for quantitative analysis. Counting results are presented in in the table 1.

TABLE 1

| | 100 μM | 50 μM | 10 μM | 0 μM | + Control | − Control |
|---|---|---|---|---|---|---|
| DET4 | 46 | 47 | 53 | 83 | 83.3 | 0 |
| monomer | | | | | | |
| Trimer + 3 | 2 | 9 | 75 | 25 | 55.6 | 0 |
| Trimer + 0 | 101 | 12 | 55 | 47 | 39 | 0 |
| PBS only | 47 | 57 | 122 | 57 | 76.3 | 0 |

While the DET4 monomer and the DNA Trimer without DET4 (Trimer+0) were not able to inhibit viral infection of target cells, the DET4-coupled DNA Trimers (Trimer+3) showed a strong decrease in the amount of foci indicating an efficient inhibition of Dengue virus infection.

### FIGURE LEGENDS

FIG. 1: Attachment of virus binding moieties to a nucleic acid scaffold.

A) schematic representation of a nanostructure according to the invention with three branches. Three influenza A-binding peptide moieties (PeB—SEQ ID NO: 1) are bound to a DNA scaffold made of three DNA strands.
B) 15% (v/v) native PAGE gel stained with SYBR® Gold Nucleic Acid Gel Stain: 1) low range DNA ladder; 2) DNA scaffold (trimer); 3) DNA scaffold (trimer) with DBCO linker; 4) DNA scaffold (trimer) with one PeB peptide moiety attached to each end (3 in total).

FIG. 2: Possible binding configurations of a nanostructure of the invention to a viral homotrimer.

The peptide moieties of the nanostructure (in this case PeB) can bind to each of the three hemagglutinins (HA) of a homotrimer, or to three hemagglutinins of three different homotrimers.

FIG. 3: Principle of the hemagglutination inhibition (HAI) assay.

A) Red blood cells (RBCs) sink to the bottom of the well of a microtiter plate.
B) When RBC-binding viruses are present in the solution, the RBCs stay in solution.
C) When the binding of the viruses to the RBCs is blocked by binding of the antibodies to the RBC-binding proteins of the viruses, the RBCs sediment again and a red dot is visible at the bottom of the well. The nanostructures according to the invention can be used in place of the antibodies.

FIG. 4: Binding of DNA-PeB nanostructures to hemagglutinin compared to PeB monomers

A) Evaluated HAI result for PeB monomers and nanostructures each made of a DNA scaffold that is attached to three PeB peptide moieties. Kᵢ^{HAI}=inhibition constant, 2HAU=2×10⁷ virus particles, 4HAU=4×10⁷ virus particles.
B) Microscale thermophoresis measurements of DNA nanostructures with three, two, one or no PeB peptide moieties (respectively PeB3 to PeB0 DNA Trimer). Only the DNA nanostructures with three PeB peptides (PeB3) strongly bind isolated hemagglutinin trimers.

FIG. 5A: Influenza Hemagglutinin protein unit and binding sites. Schematic top view (right) and front view (left) of hemagglutinin consisting of three monomers that form a trimeric protein unit.

FIG. 5B: DNA-peptide structure designed for influenza Hemagglutinin. Each arm of a symmetric DNA trimer structure carries a HA binding peptide that binds to one monomer of a HA protein.

FIG. 6A: Dengue E-protein unit & binding sites. Schematic top view of Protein E consisting of three domains: I-structural domain, II-dimerization domain, III-receptor-binding domain.

FIG. 6B: Dengue E-protein arrangement on virus surface. Schematic image of icosahedral orientation of protein E dimers at the surface of mature dengue viruses.

FIG. 6C DNA-peptide structure designed for Dengue E-protein. Schematic top view of DNA trimeric structure binding to protein E dimers. Due to its asymmetric shape, peptides arranged on the DNA structure can bind to domain III of two different protein E dimers.

FIG. 7A: RSV F-protein unit & binding sites. Schematic front view of RSV-F monomers arrange to a trimer on the viral surface. Binding sites are located on the upper sides of the monomers.

FIG. 7B DNA-peptide structure designed for RSV F-protein. Schematic front view of DNA trimeric structure capable of binding to the outer sides of the receptor monomers due to elongated single-stranded DNA overhangs.

FIG. 8: SPR to analyse binding of inactivated influenza A virus (analyte) to different DNA constructs. DNA constructs (ligands) were labelled with biotin and conjugated to a streptavidin chip. A) Comparison of no ligand (flow cell 1) and DNA construct carrying a biotin label (star shaped) and 1×PeB (ravel-shaped) (flow cell 2). Binding of viruses to PeB-conjugated DNA is clearly visible whereas viruses did not bind to the unconjugated streptavidin chip. B) Comparison of DNA 4-arm structure without peptides (flow cell 3) and DNA 4-arm structure with 3×PeB (flow cell 4). Viruses did not bind to DNA structures without peptide. Compared to the simple DNA double-strand (flow cell 2), the binding of viruses to DNA 4-arm structures carrying 3×PeB (flow cell 4) has a slower dissociation time and thus seems to be more stable.

FIG. 9: Design of new DNA constructs. Schematic image of DNA trimer variants. Starting from a symmetric rigid DNA construct, alterations of arm length and insertion of unpaired bases in the middle lead to constructs with different flexibility, symmetry and size.

FIG. 10A: MST data for structure Z_3×PeB and Z without PeB. Constant concentration of inactive influenza A viruses were incubated with different concentrations of fluorescently-labelled DNA trimeric constructs. As control, constructs without PeB were analysed. Viruses do not bind to DNA structures without peptides (concentration changes do not results in different values) but clearly to DNA structures with 3×PeB in a concentration dependent manner.

FIG. 10B: MST data for structure W. 3×PeB and W without PeB. Constant concentration of inactive influenza A viruses were incubated with different concentrations of fluorescently-labelled DNA trimeric constructs. As control, constructs without PeB were analysed. Viruses do not bind to DNA structures without peptides (concentration changes do not results in different values) but clearly to DNA structures with 3×PeB in a concentration dependent manner.

FIG. 11: HAI assay using N_3×PeB and PeB. Concentration of viruses and blood was kept constant and DNA structures “N” carrying three PeB moieties were added as dilution series. RBCs forming a dot on the bottom of the well can be seen for high concentrations of PeB-DNA scaffolds (left part, highest concentration 9.5 μM). At lower concentrations (right part of the plate), the viruses are not hindered anymore and can bind to RBCs. As a result, the RBCs stay in solution. Due to bad image quality, the clearest differences can be seen on the far left row. In addition, PeB alone was tested in a dilution series, too (highest concentration on far left side was 250 μM).

FIG. 12: HAI assay using X_3×PeB, N_3×PeB, Y_3×PeB. Concentration of viruses and blood was kept constant and DNA scaffolds carrying three PeB moieties were added as dilution series. RBCs forming a dot on the bottom of the well can be seen for high concentrations of PeB-DNA scaffolds (left part). At lower concentrations (right part of the plate), the viruses are not hindered anymore and can bind to RBCs. As a result, the RBCs stay in solution. Due to bad image quality, one needs to compare the sizes of the dots to see whether agglutination happened. “Y unmod” resembles unmodified DNA nanostructures as controls. Whereas X_3×PeB and N_3×PeB hinder the viruses from binding to RBCs; Y_3×PeB as well as Y unmodified do not hinder the viruses. Wells covered with X represent invalid wells.

FIG. 13: HAI assay using V_3×PeB, U_3×PeB, W_3×PeB, Z_3×PeB. Concentration of viruses and blood was kept constant and DNA scaffolds carrying three PeB moieties were added as dilution series. RBCs forming a dot on the bottom of the well can be seen for high concentrations of PeB-DNA scaffolds (left part). At lower concentrations (right part of the plate), the viruses are not hindered anymore and can bind to RBCs. As a result, the RBCs stay in solution. Due to bad image quality, one needs to compare the sizes of the dots to see whether agglutination happened (especially influenza+RBCs only). All structures at higher concentrations bind viruses and thus RBCs can settle.

FIG. 14A: Infection inhibition assay using MDCKII cells. This is a microscopic image of MDCKII cells. The cells form a healthy monolayer. No cytopathic effect (CPE) is seen.

FIG. 14b: Infection inhibition assay using MDCKII cells. This is a microscopic image of MDCKII cells infected with influenza viruses. The cells begin to die. A cytopathic effect (CPE) (rounding up and small aggregations of the cells) is clearly seen.

FIG. 14C: Infection inhibition assay using MDCKII cells. This is a microscopic image of MDCKII cells with influenza viruses and Trimer “N” with 3×PeB at 11 μM—Far less CPE is observed. The cells still mostly form a monolayer and are healthy. Virus infection was inhibited. Compared to monomeric peptide alone, see FIG. 14E, a much better inhibition at 50× less concentration of the construct can be observed.

FIG. 14D: Infection inhibition assay using MDCKII cells. This is a microscopic image of MDCKII cells with influenza viruses and Trimer “N” without PeB at 11 μM. Here the DNA trimer only without any anti-viral peptide is present. CPE is seen. The DNA Trimer alone cannot inhibit the infection.

FIG. 14E: Infection inhibition assay using MDCKII cells. This is a microscopic image of MDCKII cells with influenza viruses and PeB 500 μM (no DNA construct). Here the anti-viral peptides only are present. The peptides are in a 500× higher concentration as the trimer-PeB in FIG. 17c—CPE is seen. The peptide alone is not enough to inhibit the infection.

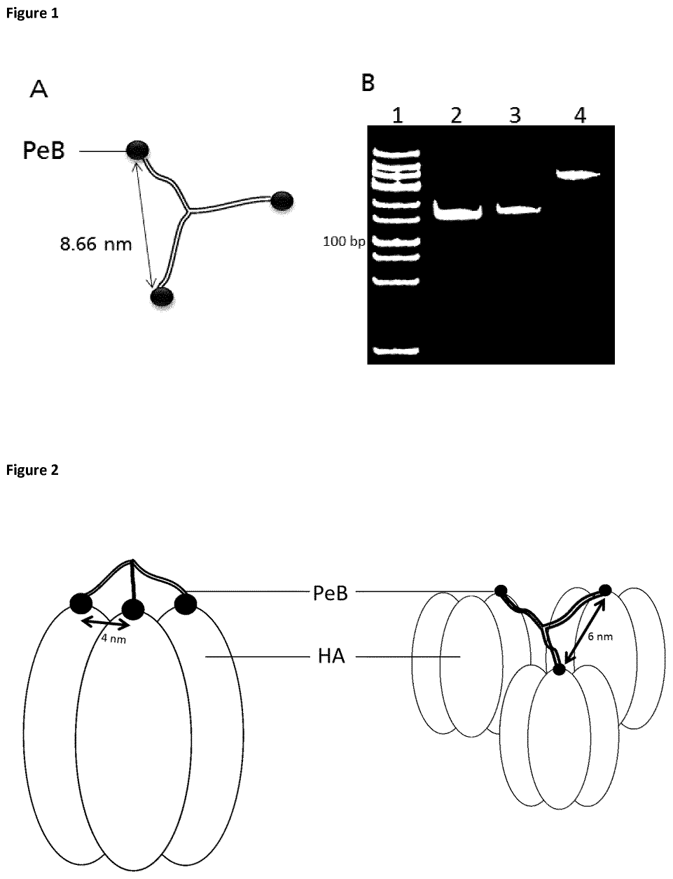

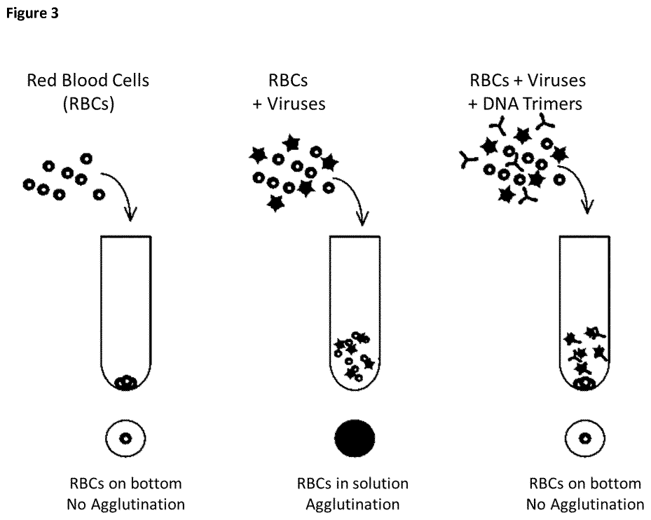

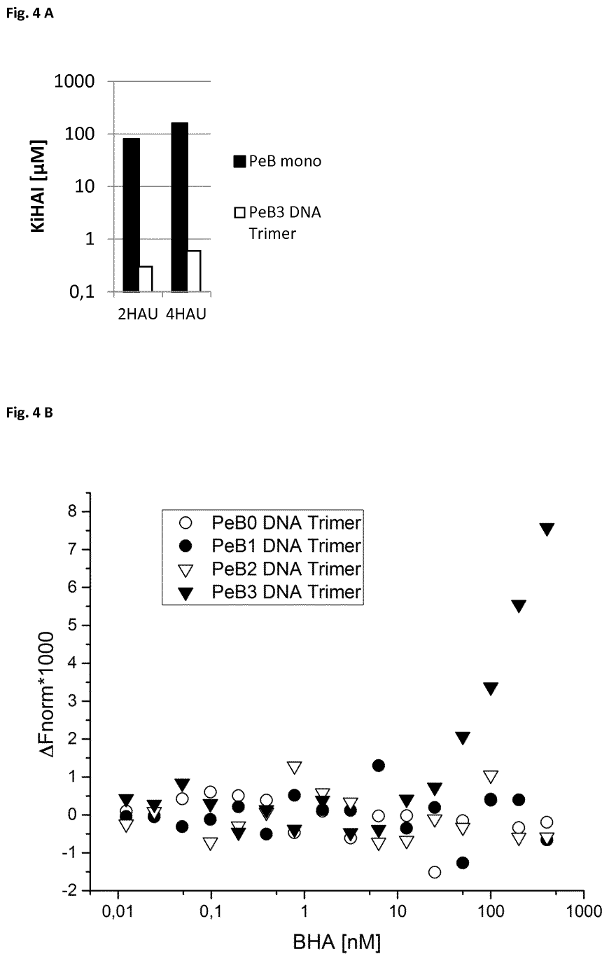

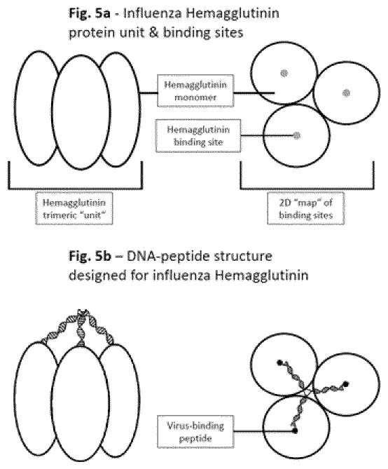

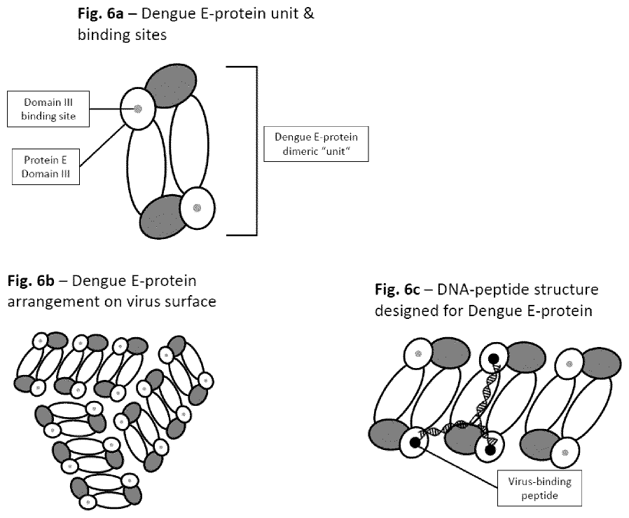

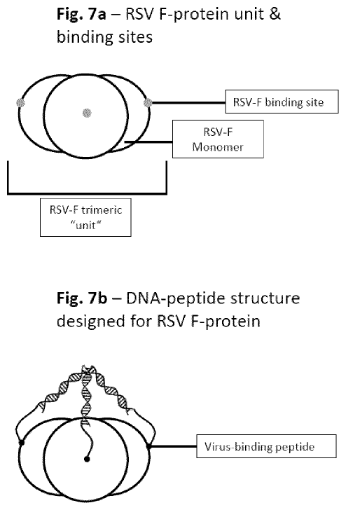

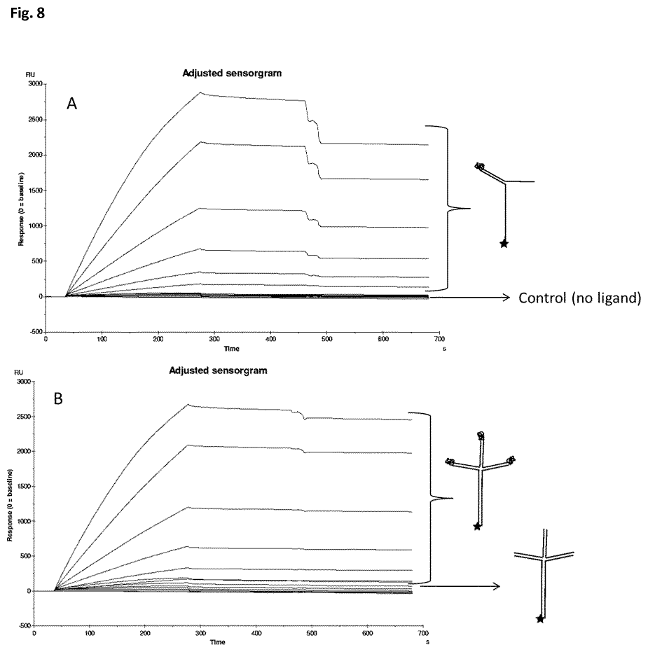

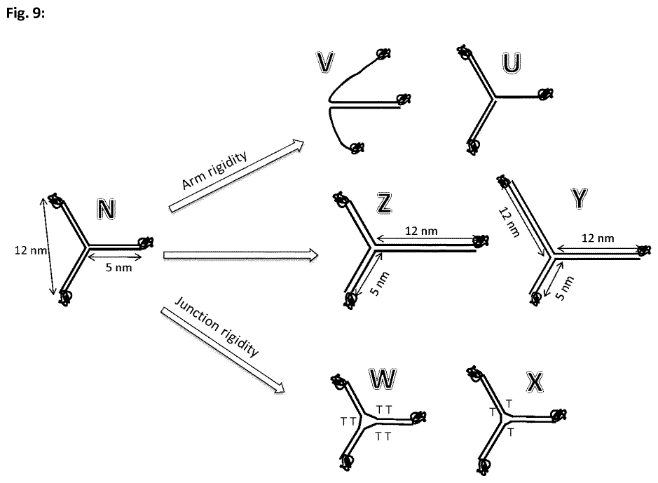

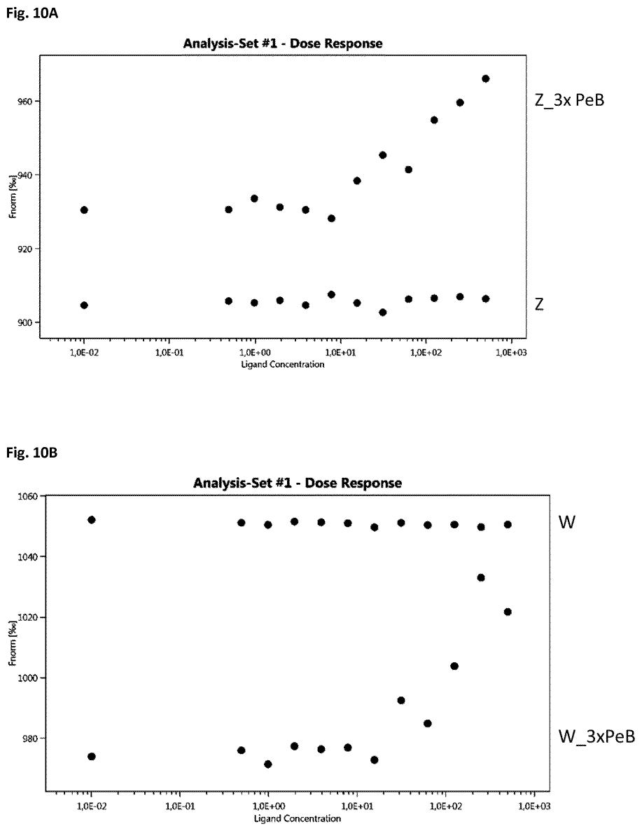

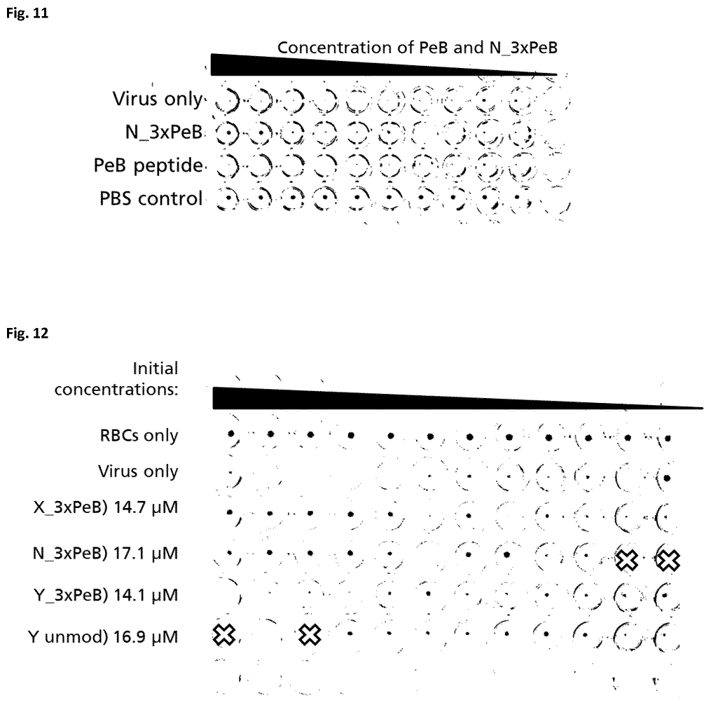

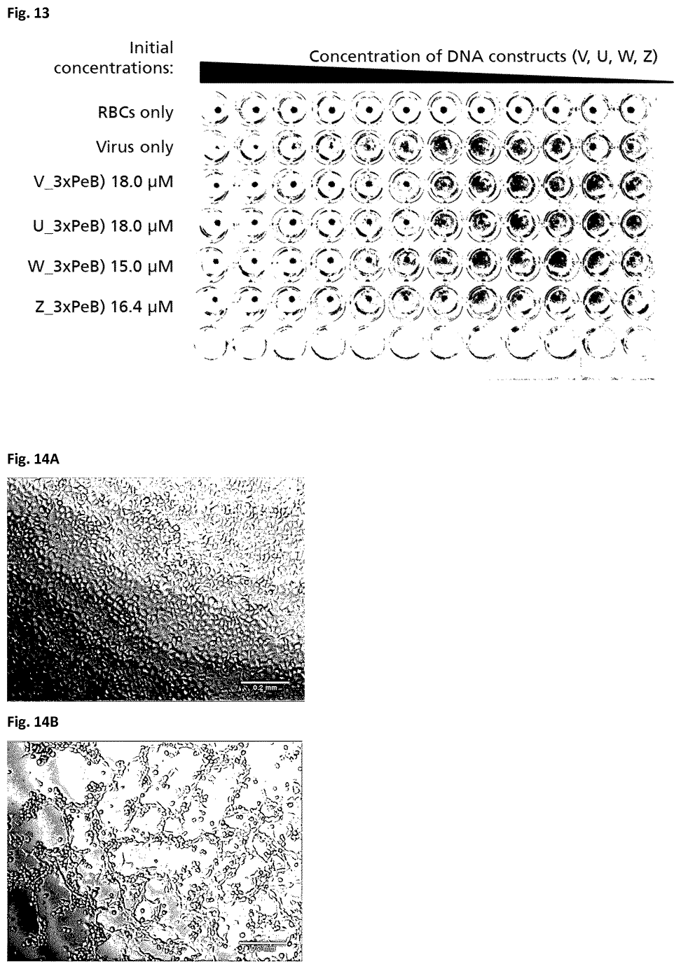

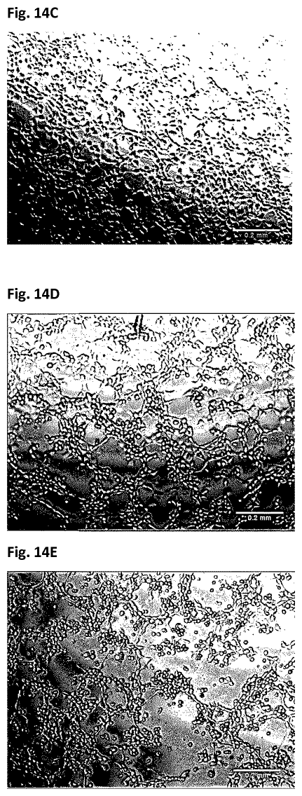

What is claimed is:

1. A nanostructure comprising:
a) a nucleic acid scaffold; and
b) at least two peptide moieties, wherein the at least two peptide moieties specifically bind to a molecule expressed on the surface of a virus and are attached to the nucleic acid scaffold,
wherein the nucleic acid scaffold is selected from the group of:
i) a linear nucleic acid scaffold, wherein the at least two peptide moieties are each attached at or near different ends of the nucleic acid scaffold; and
ii) a branched nucleic acid scaffold, wherein the at least two peptide moieties are each attached to a different branch of the scaffold.

2. The nanostructure of claim 1, wherein the nucleic acid scaffold comprises double stranded nucleic acids, wherein the double stranded nucleic acids are over a sufficient length to remain stable in physiological conditions.

3. The nanostructure of claim 2, wherein each of the strands of the double stranded nucleic acids is DNA, RNA, LNA, PNA, or XNA.

4. The nanostructure of claim 1, wherein the at least two peptide moieties each bind to a molecule expressed on the surface of a target virus with a K_{D} 500 μM or less under physiological conditions.

5. The nanostructure of claim 1, wherein the at least two peptide moieties bind to the same viral molecule with a K_{D} of 500 μM or less under physiological conditions.

6. The nanostructure of claim 1, wherein the at least two peptide moieties attached to the nucleic acid scaffold are the same.

7. The nanostructure of claim 1, wherein each of the at least two peptide moieties attached to the nucleic acid scaffold binds to a molecule expressed on the surface of a virus selected from the group of the orthomyxoviridae, filoviridae, retroviridae, coronaviridae, togaviridae, flaviviridae, and pneumoviridae.

8. The nanostructure of claim 1, wherein the at least two peptide moieties attached to the nucleic acid scaffold bind to the influenza A hemagglutinin.

9. The nanostructure according to claim 8, wherein the at least two peptide moieties comprise SEQ ID NO: 1.

10. The nanostructure of claim 1, wherein the at least two peptide moieties attached to the nucleic acid scaffold bind to domain III of DENV-2 E protein.

11. The nanostructure according to claim 10, wherein the at least two peptide moieties comprise SEQ ID NO: 5.

12. The nanostructure of claim 1, further comprising at least three peptide moieties, wherein the nucleic acid scaffold is branched and the at least three peptide moieties are each attached to a different branch of the nucleic acid scaffold.

13. The nanostructure of claim 12, wherein the at least three branches of the nucleic acid scaffold emanate from a single junction.

14. A nanostructure according to claim 1 for use as a medicament.

15. The nanostructure of claim 14 for use in the treatment of a viral infection or in the prophylactic treatment of a viral infection.

16. A nanostructure according to claim 1 for use in diagnosing a viral infection.

17. A method of detecting whether a specific virus is present in a sample comprising the steps of:
a. adding to the sample an appropriate amount of the nanostructure of claim 1, wherein the at least two peptide moieties of the nanostructure bind under physiological conditions to at least one molecule expressed on the surface of the virus to be detected;
b. incubating a mixture obtained in a. under physiological conditions; and
c. detecting whether the nanostructure has bound to the surface of the virus,
wherein the binding of the nanostructure to the virus is indicative of the presence of the virus in the sample.
